(19) United States
(12) Patent Application Publication
(10) Pub. No.: US 2025/0066844 A1
(43) Pub. Date: Feb. 27, 2025

(54) COMPOSITION AND METHOD FOR AMPLIFYING NUCLEIC ACID SEQUENCES

(71) Applicant: BFORCURE, Montreuil (FR)

(72) Inventors: Maël LE BERRE, Montreuil (FR); Christophe PANNETIER, Montreuil (FR); Francesco VILLA, Montreuil (FR); Maryna MURLYKINA, Montreuil (FR); Julien CROZZOLO, Montreuil (FR)

(73) Assignee: BFORCURE, Montreuil (FR)

(21) Appl. No.: 18/722,223

(22) PCT Filed: Dec. 13, 2022
(86) PCT No.: PCT/FR2022/052335

§ 371 Date: Jun. 20, 2024

(30) Foreign Application Priority Data
Dec. 22, 2021 (FR) FR2114256

(51) Int. Cl. C12Q 1/686

(52) U.S. Cl. CPC C12Q 1/686

(57) ABSTRACT

A composition is disclosed for the amplification of nucleic acid sequences, which is intended to be mixed with a biological sample containing one or more target nucleic acid sequences and with primers specific for the target nucleic acid sequence(s), and fluorescent probes for revealing an amplification of the target nucleic acid sequence(s). The composition includes reagents and enzymes for nucleic acid application. The composition also includes a polymerizable reagent, which polymerizes under the influence of a stimulation chosen from a heat stimulation and a light stimulation, so as to form a hydrogel which locally encapsulates each target nucleic acid sequence when the composition is exposed to the stimulation. A method using said composition is also disclosed.

The invention relates to a composition for amplifying nucleic acid sequences, as well as to a method for amplifying nucleic acid sequences which uses this composition. The invention also relates to a device especially designed for use of the composition.

More generally, the field of the invention is that of detection of nucleic acid sequences by an amplification reaction, such as PCR or any other in vitro amplification method, in a controlled-scattering medium compatible with such methods.

In molecular biology, it is regularly necessary to identify with a great level of accuracy and confidence the presence of one or more nucleic sequence(s) of interest (or target nucleic sequences) in a sample. Thus, it is possible to measure the relative or absolute concentration of these sequences of interest.

For example, the target sequences may consist of DNA sequences such as gene fragments present in a specific manner in the genome of pathogens for humans or animals. Consequently, the target sequences allow identifying the pathogenic agent that carries them, or characterising their pathogenic power or their ability to withstand one or more antibiotic(s) or one or more antiviral molecule(s). The target sequences may also consist of DNA sequences located outside the genes, such as telomeres, transposons or retrotransposons, in particular. Some of the target sequences may be distinguished from one another by one or more point mutation(s), i.e. a change of one base by another (“single nucleotide polymorphism” in English), or the addition or deletion of a base or of several bases. The target sequences may also be made of RNA, like, for example, gene transcripts, microRNAs, non-translated RNAs or viral RNAs originating from RNA viruses and retrotransposon transcripts. In some situations, the target sequences are artificial DNA sequences used as barcodes or “tag”. This is in particular the case of sequences covalently bonded to antibodies used in so-called immuno-PCR methods, or sequences integrated into padlock probes (“padlock probes” in English).

The target nucleic sequences may be in different states within the sample. Thus, the sequences may be free in the sample, possibly after a step of treatment of this sample, or included in biological particles such as viruses, prokaryotic or eukaryotic cells, or organelles of these such as mitochondria. In this case, these nucleic sequences are generally selected so that they allow identifying the biological particles, or characterising therein a genotype or a particular property.

Independently of the state of the sequences, when they are present in an amount that is too low to be characterised directly, a method commonly used to detect them consists in carrying out an in vitro amplification of these sequences in order to obtain a sufficient amount to enable characterisation thereof. This amplification is carried out using a mixture of oligonucleotide primers located in the target sequences. The addition of one or more oligonucleotide probe(s) conjugated to a fluorophore conventionally allows characterising the presence or absence of the target sequences as well as their amount.

A well-known amplification method is the polymerase chain reaction PCR (“Polymerase Chain Reaction” in English). PCR is an in vitro exponential amplification process. This is the most commonly used amplification process at the time being. Besides PCR, there are other alternative processes such as the LCR process (“Ligation Chain Reaction” in English), the NASBA process (“Nucleic Acid Sequence-Based Amplification” in English), the TMA process (“Transcription Mediated Amplification”), the LAMP process (“Loop-Mediated Isothermal Amplification” in English), the SDA process (“Strand Displacement Amplification” in English) and the so-called RCA rolling circle amplification (“Rolling Circle Amplification” in English).

PCR and the other in vitro amplification reactions are usually carried out in a liquid reaction medium containing the different reagents. These typically consist of primers, fluorescent probes specific for the target sequences, enzymes for amplifying nucleic acids, optionally additives, and of the sample(s) to be analysed including the target sequence(s). During the amplification reaction, the reagents as well as the products resulting from the reaction, so-called amplicons, diffuse freely into the liquid volume of the reaction medium. This free diffusion in the liquid reaction medium is at the origin of several drawbacks which are prohibitive for some applications.

Indeed, in some applications, the target sequences are present in small amounts in the samples (1 to 10 molecules for example). Hence, a procedure should be provided for to ensure a very highly sensitive detection. This is in particular the case of the in vitro diagnosis of particular nucleic sequences in a blood sample of a person, which could for example indicate the presence of sepsis, of a tumour, or a genetic disease in a foetus (detection in a so-called liquid biopsy). Even though the amplification methods might theoretically be capable of detecting one single molecule of a target sequence, in practice, the diffusion of the amplicons into the reaction mixture has the effect of delaying the time point of the reaction at which the intensity of the signal emitted by the amplicons and measured by the detector of the used device locally exceeds the level of the background noise of the detector. This delay could be such that the amplicons are finally not detected during the reaction, resulting in a false negative result.

Free diffusion also poses a problem in applications requiring the detection of a plurality of target sequences in the same reaction mixture. This is in particular the case in applications related to the diagnosis of infectious diseases wherein it is essential to simultaneously identify the pathogenic agent(s) involved, but also their genotype of resistance to antibiotic or antiviral molecules. While a solution for meeting this requirement might consist in detecting each sequence of interest in a monoplex test, it seems that this solution is restrictive and that it might in particular be desirable to carry out the detection of all of the sequences of interest in one single reaction. The reasons may be the emergency, the small size of the sample, the low concentration of the target sequences in the sample, or the cost of the test (reagents, consumables, occupancy rate of the automaton, etc.). Yet, the free diffusion of the amplicons derived from the target sequence predominantly present in the sample has the effect of interfering with the amplification of the other minority target sequences in this sample. This is due in particular to the competition for the reagents necessary for the amplification reaction. Thus, there is a permanent risk that one or more minority target sequence(s) are not detected in the sample.

In other applications, it is necessary to measure the amount of target sequence(s) present in a sample. Thus, there is a need for an accurate quantification or measurement of a distribution. More particularly, it is sometimes necessary to ensure that the presence of a target sequence present in large amounts in a sample does not interfere with the measurement of the concentration of other target sequences present in less amounts (for example 10, 100, or 1,000 times less abundant). Such a measurement is impossible when the target sequences and their amplicons diffuse freely in the reaction mixture.

To overcome some of the aforementioned problems, the prior art proposes different approaches.

In particular, there is the approach of an in situ PCR amplification. This approach is useful when the target sequences are included in a cell. Thus, the in situ PCR includes a step of amplification of DNA or of local RNA in order to improve the sensitivity and specificity performances of the hybridisation. In this technique, the amplification may be carried out in solution. For this purpose, the cell(s) should be fixed, and made permeable so as to enable the PCR reagents to enter the cell. The amplification may also be carried out on histological sections fixed in the formol (U.S. Pat. No. 5,364,790, U.S. Pat. No. 5,538,871 and U.S. Pat. No. 5,804,383). The main drawback relates to the permeabilisation of the cells in order to cause the reagents to penetrate therein. This permeabilisation enables the amplicons to diffuse. This makes the methods for characterising the amplified products in liquid phase inapplicable. Hence, it is necessary to manipulate the sample after amplification in order to carry out the step of detecting the amplicons fixed in the cells. This lengthens the duration and increases the complexity of the reaction and analysis thereof. Furthermore, it is an expensive operation for which the risks of cross-contamination are increased. For these reasons, in situ PCR did not in particular not allows meeting the needs of multiple genotyping (Uhlmann et al. 1998).

Another approach is a method so-called digital amplification or digital PCR. In this method, the reaction mixture is divided into a large number of distinct compartments of small volume (for example in the range of one picolitre). The compartments are selected so that each compartment contains only at most one molecule of the target sequence. This physical compartmentalisation may be achieved, for example, by the production of an emulsion of reaction medium droplets in an oily liquid thanks to a microfluidic system. It may also be obtained by filling a microfluidic device consisting of a large number of small-volume cells. Irrespective of the means by which it is obtained, this compartmentalisation allows carrying out a large number of completely distinct amplification reactions, without passage of a reaction mixture from one compartment to another. The compartments in which an amplification reaction is obtained are detected and counted. This count allows carrying out an accurate quantification of the number of molecules of target sequences present in the sample. Thus, the digital PCR allows quantifying the presence of a few target sequences in one single reaction [Sensors (Basel) 2018, 18 (4), 1271; J. Med. Virol. 2021, 93 (7), 4182-4197]. However, carrying out a digital PCR reaction requires complex and expensive equipment. In particular, the compartmentalisation of the reaction mixture requires complex manipulations. In addition, digital PCR does not allow preserving the advantages of the so-called real-time PCR (with the measurement of a Ct in particular).

A promising approach is to carry out a PCR in a gel matrix and to measure PCR products generated at each cycle. The PCR may be performed inside a polyacrylamide gel (PAM) (Anal. Biochem. 2006, 356, 300-302) or a PEG derivative. Thus, the reaction generates distinct molecular colonies which are sometimes so-called “polonies” (P. Blainey et al. U.S. Pat. No. 10,487,354; Huang et al. US2019/0202268; Huang et al. US2019/0202268; G. M. Church et al. U.S. Pat. No. 6,485,944; A. B. Chetverin et al. EP1999268; A. B. Chetverin et al. U.S. Pat. No. 6,001,568). The main advantages of these matrices are as follows: i) with a suitable mesh size, they delay the diffusion of the linear molecules so that the target molecules and their amplification products remain located at close proximity; ii) the number of formed polonies is proportional to the number of target molecules initially present in the sample, which facilitates quantification in a similar manner as digital PCR; and iii) it is possible to fix at least one of the primers to the gel by covalent bonding. Nonetheless, a major drawback of the gels of the prior art is their incompatibility with applications outside the laboratory. For example, the techniques of the prior art are unsuitable for rapid diagnostic applications. This in particular due to the gelation process which is not controllable neither spatially nor temporally. In addition, in the case of PAM gels, the target molecules and the amplification reagents should be incorporated into the gel already formed after having been rinsed with a buffer in order to extract therefrom the acrylamide monomers that have not polymerised due to their high inhibitory effect on the PCR amplification reaction.

The present invention improves the situation.

Thus, the invention relates to a masterbatch composition for the amplification of nucleic acid sequences, intended to be mixed with a biological sample containing one or more target nucleic acid sequence(s) and with primers specific for the target nucleic acid sequence(s), as well as fluorescent probes for revealing an amplification of the target nucleic acid sequence(s), the composition comprising reagents and nucleic acid amplification enzymes, characterised in that the composition further comprises a polymerisable reagent, which polymerises under the influence of a stimulation selected from among a thermal stimulation and a light stimulation, SO as to form a hydrogel locally encapsulating each target nucleic acid sequence when said composition is exposed to said stimulation.

Thus, the invention allows carrying out amplification reactions of nucleic acid sequences in a medium wherein the diffusion of the constituents is controlled. Thus, the invention allows locally encapsulating the target nucleic acid sequence(s). This results in a large number of advantages related in particular to the detection of the nucleic acids and/or to the localised control of the amplification reactions.

In one embodiment, the polymerisable reagent is a photosensitive reagent which polymerises when exposed to light, preferably ultraviolet light. This allows accurately controlling the initiation of the controlled polymerisation of the composition of the invention.

The polymerisable reagent may also be a heat-sensitive reagent which will be polymerised when exposed to a temperature above a threshold temperature. One advantage of this embodiment is that it is possible to directly use the temperature domains used in the nucleic acid amplification processes, isothermal or not.

Preferably, the polymerisable reagent includes already polymerised chains, advantageously, only the terminal function of which is reactive to more easily control the size of the pores of the gel. These already polymerised chains may be selected from among water-soluble polymers that do not, or barely, inhibit PCR (for example PEGs, PAMs, biopolymers, etc.).

In general, the polymerisable reagent may be a poly (ethylene glycol) with multiple arms (or PEG with multiple arms, or multi-arm PEG).

Still more generally, the polymerisable reagent is advantageously pre-polymerised and/or water-soluble and biologically inert (so as not to interfere with the amplification reaction) and/or able to polymerise (with an activatable function or naturally radical, for example).

Thus, in a particular embodiment, the polymerisable reagent is selected from among the class of multi-arm PEGs (multi-arm PEGs) and derivatives thereof.

The polymerisable reagent may be selected from among 4-Arm-PEG-SH, 4 Arm-PEG-norbornene, 2-Arm-PEG-SH, 8-Arm-PEG-norbornene (tripentaerythritol), nitrocinnamate-based 8-Arm-PEG or a mixture thereof. These are photosensitive reagents which are polymerisable by light at a selected wavelength and thus contribute to the accuracy of the controlled polymerisation of the composition of the invention.

Advantageously, the photosensitive polymerisable reagent is a mixture of 2 Arm-PEG-Thiol and of 8 Arm-PEG-norbornene or of 4 Arm-PEG-Thiol and of 4 Arm-PEG-norbornene in the presence of the lithium-phenyl-2, 4, 6-trimethylbenzoylphosphinate (LAP) photo-initiator.

The polymerisable reagent may be 4 Arm-PEG-acrylate. This is a reagent which polymerises in the presence of a thermo-initiator when the latter is exposed to a given temperature.

Advantageously, the heat-sensitive polymerisable reagent is a mixture of 4 Arm-PEG-Acrylate in the presence of the thermo-initiator 2, 2′-azobis [2-(2-imidazolin-2-yl) propane dihydrochloride].

More generally, the photosensitive polymerisable reagent may be selected from among water-soluble polymers bearing unsaturated functions (norbornene, nitro-cinnamate, etc.) or not (thiols) or a mixture thereof. These are reagents capable of forming reactive intermediate species, preferably radicals or radical ions, where appropriate, in the presence of an activator (the polymer itself or a photo-initiator), by application of a light at a wavelength comprised between 200 and 800 nm, preferably between 300 and 450 nm.

Furthermore, the heat-sensitive polymerisable reagent may be selected from among water-soluble polymers bearing alkene (acrylate, acrylamide, etc.) functions or a mixture of activated unsaturated functions (maleic anhydride, maleimide, etc.) with conjugated 1, 3-dienes (furan, thiophene, etc.). These are reagents capable of forming reactive intermediate species, preferably radicals or radical ions, or participate in cycloaddition reactions by application of a threshold temperature comprised between 30 and 95° C., preferably between 50 and 80° C.

In one embodiment, the polymerisable reagent is present in a content of 2% to 25%, preferably 5% to 15%, by weight per unit volume (w/V %).

In one embodiment, the composition further

comprises a polymerisation initiator agent. It may consist of a photo-initiator or a thermo-initiator. The initiator is present in a content from 0.02% to about 0.1% by weight per unit volume (w/V). preferably, the photo-initiator is lithium phenyl-2, 4, 6-trimethylbenzoylphosphinate (LAP), and the thermo-initiator is the 2, 2′-azobis [2-(2-imidazolin-2-yl) propane dihydrochloride]. The agent allows facilitating the initiation of the polymerisation and thus contributes to the controlled polymerisation of the composition of the invention.

Furthermore, the composition may include ab inito a developer of the presence of nucleic acid sequences, preferably fluorescent. The developer may be a fluorescent intercalator of the nucleic acid or a fluorescent probe specific for a sequence combined with a fluorescence quencher. The developer offers better detection of the amplified nucleic acids during or at the end of the amplification reaction.

The composition may also include ab inito primers specific to the target nucleic acid sequence(s). This allows in particular preparing masterbatches especially designed so as to identify a known pathogen. In this embodiment, it is also advantageous to directly include the specific probe(s) in the composition to reveal the presence of the amplified target nucleic acid sequences.

In a preferred embodiment of the invention, the composition of the invention remains liquid before application of said stimulation for a time period comprised between several hours and several months, preferably between several days and several months, and said composition forms the hydrogel after application of said stimulation within a time period shorter than or equal to 10 minutes, preferably shorter than or equal to 1 minute.

Of course, the composition is stable over time, i.e. throughout the duration. which the composition remains liquid (or very slightly viscous) and beyond, so as to function as a composition for amplifying nucleic acid sequences. By “stable”, it should be understood in particular the fact that the reagents do not (or barely) degrade under normal storage conditions (for example in the refrigerator, at room temperature, protected from light, etc.).

The invention also relates to a method for amplifying nucleic acid sequences comprising the following steps: (i) providing the composition of the invention; (ii) mixing said composition with a biological sample to be analysed (iii) applying a stimulation selected from among a thermal stimulation and a light stimulation so as to polymerise the mixture of step (ii) to obtain a hydrogel; (iv) exposing the hydrogel to an acid sequence amplification reaction.

Step (ii) may include mixing the composition with primers specific to the target nucleic acid sequence(s) and/or to specific probes to reveal the presence of the amplified target nucleic acid sequences.

In one embodiment, the provision step (i) is maintained for a time period comprised between several hours (for example 1 to 5 hours) and several months (for example 1 to 18 months), preferably between several days (1 to 15 days) and several months (2 to 6 months).

A key point of the method of the invention is that the polymerisation in step (ii) begins only after application of a light or thermal stimulus. Thus, the light stimulus may consist in the application of a light with a wavelength comprised between 200 nm and 800 nm, preferably between 300 nm and 450 nm; and the thermal stimulus may consist in the application of a threshold temperature comprised between 30°° C. and 95° C., preferably between 50°° C. and 80°° C. for a time period shorter than 10 min.

Advantageously, the time period between the provision of the composition and the application of the stimulus is longer than 1 h, preferably longer than 1 month, which avoids preparing the composition just before use thereof.

In other words, the composition of the invention can be stored for a period longer than 1 month and which could last up to six months and beyond.

Better still, each step of the method may be characterised by a duration: (t₀) for storage of the composition containing the polymer(s) the and amplification reagents, up to several months depending on its composition and on the storage conditions; (t₁) for handling the same composition once it has been stored, and brought into the presence of the target without gelling under ambient conditions, which could last up to several hours; and (t₂) for gelling after application of a stimulus (light or temperature), being shorter than 10 minutes.

In other words, the composition including the polymerisable agent is stable for a very long period of up to several months. This is very advantageous for storage conditions. Afterwards, the composition could be mixed with the target sequence, without the polymerisation process directly converting the solution into gel. In practice, the gelation progresses very slowly and the formation of a gel is observed only after several hours. This results in another great advantage which is that there is no need to hurry up manipulations in the laboratory. In particular, this allows preparing several samples in parallel. Thus, it is possible to initiate the polymerisation of several samples in parallel in the same stimulus operation. The gelation of all of the samples lasts only a few minutes. It follows that the target sequence amplification process could then be started for all samples in parallel. Thus, in a particular embodiment, the hydrogel is obtained within a time period shorter than or equal to 10 minutes, preferably shorter than or equal to 1 minute after the application of the stimulation of the method of the invention.

The hydrogel formed by the composition of the invention encapsulates the amplification reagents as well as the target nucleic acid sequence(s). It follows that amplification reactions of nucleic acid sequences are carried out locally at distinct locations of the gel. The amplification products remain encapsulated in the hydrogel. In other words, both the diffusion of the constituents of the composition and the diffusion of the products of the amplification reaction are controlled. This results in a large number of advantages related in particular to the analysis and detection of nucleic acids.

In one embodiment, the amplification reaction is a polymerase chain reaction.

In one embodiment of the invention, the polymerisation is initiated by exposure of the composition to ultraviolet light (when the polymerisable agent is photosensitive) or a threshold temperature (when the polymerisable agent is heat-sensitive). This polymerisation may be related to a reaction selected from the group consisting of a bio-orthogonal thiol-ene reaction and a dimerisation reaction.

Other advantages and features of the invention will appear upon reading the detailed description hereinafter and in the appended drawings, wherein:

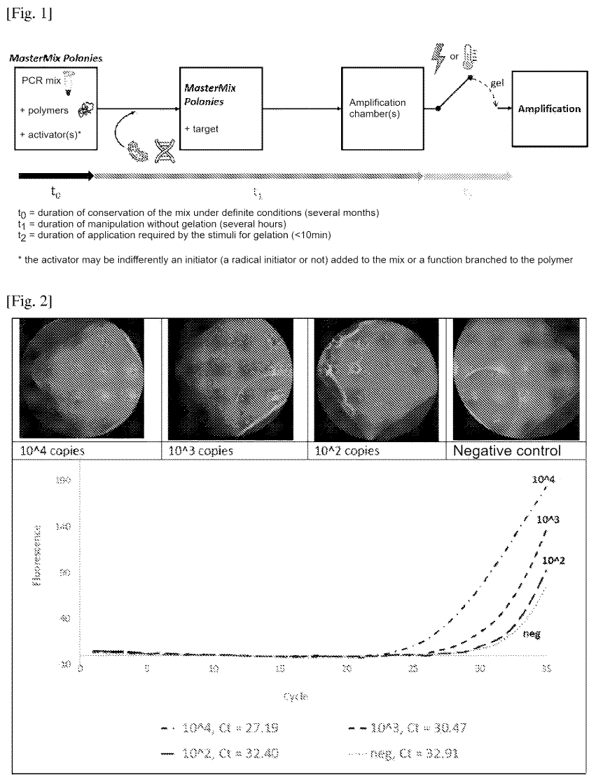

FIG. 1 shows a principle figure of the invention;

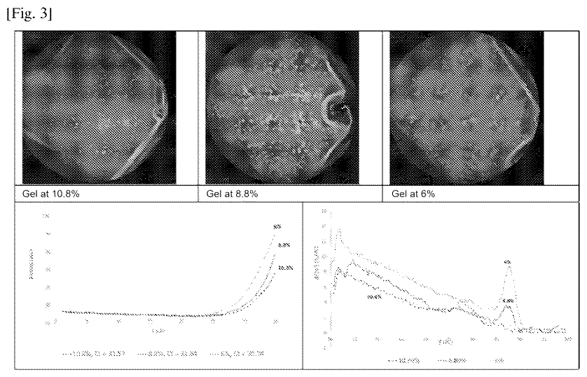

FIG. 2 shows photographs of hydrogels formed by the composition of the invention with a photosensitive agent having initial concentrations of different target sequences, as well as a graph corresponding to the PCR data of a first embodiment of the invention;

FIG. 3 shows two photographs of a hydrogel formed by the composition of the invention of the first embodiment; and

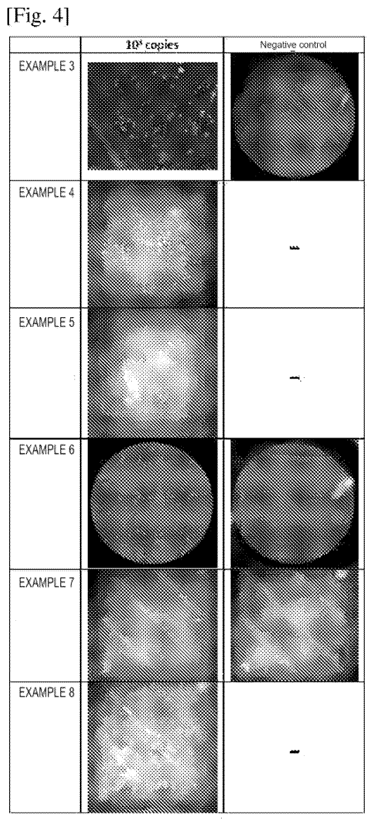

FIG. 4 shows photographs of hydrogels formed by different embodiments of the composition according to the invention having an identical initial concentration of target sequences (10³ copies).

The figures, tables and the description hereinafter contain, essentially, elements of a certain nature. The figures and tables form an integral part of the description, and could therefore not only serve to better understand the present invention, but also contribute to definition thereof, where appropriate.

In general, the present invention relates to compositions including a polymerisable agent. The compositions are biocompatible and have a low viscosity at room temperature. Thus, the compositions of the invention allow mixing target molecules or target cells at room temperature with reagents for amplifying nucleotide sequences. This has the advantage that manipulations by laboratory operators could be done in the usual manner. The compositions of the invention have the particularity of being able to rapidly form a homogeneous network hydrogel by means of the polymerisable agent. The polymerisable agent triggers a polymerisation of the composition when exposed to a triggering factor, such as a light stimulus or a threshold temperature. The composition of the invention ensures a sufficient sensitivity 4 the PCR, in particular thanks to the analysis of images of the gel. When the composition of the invention is in a gel state, it could then be subjected to a nucleic acid amplification reaction.

The composition of the invention in the gel state has a matrix capable of withstanding rapid changes in temperature generally required during the amplification reactions. Furthermore, the composition in gel form preserves its structural integrity under the effect of the different physical and/or chemical constraints applied thereto during the nucleic acid amplification processes.

An objective of the invention is to make gel matrices with a homogeneous structure. The gel has suitable meshes which drastically limit, and even prohibit, the diffusion of the target molecules and of the amplification products, while enabling a diffusion of the amplification reagents to carry out the nucleic acid amplification reaction(s). This objective is achieved by the main claim appended to the present description of the invention.

Conventionally, hydrogels are networks of three-dimensional (3D) crosslinked polymers, capable of absorbing and retaining large amounts of water. Because of their adjustable properties and their versatile manufacturing methods, hydrogels are used in a wide range of biomedical applications and techniques. For example, mention may be made of tissue engineering and regenerative medicine or the treatment of wastewater or robotics. In general, hydrogels are formed from monomers or chains of pre-polymers by covalent and/or non-covalent bonds such as hydrogen bonds, electrostatic interactions, so-called host-guest complexations (“host-guest” in English) and combinations thereof (W. Wang, R. Narain, H. Zeng. Polymer Science and Nanotechnology, Fundamentals and Applications, 2020, Pages 203-244).

The prior art describes gel matrices used for nucleic acid amplification. These gel matrices are formed in polymerisation reactions according to two basic mechanisms: chain polymerisation and step-by-step polymerisation.

The chain polymerisation (“chain growth polymerisation” in English) consists in forming a polymer from unsaturated monomer molecules (in particular acrylamide, methacrylamide, acrylic acid, methacrylic acid, bis-acrylamide, styrene, etc.). This reaction type requires an initial activation of the unsaturated bond(s). In other words, it is necessary to form a reactive species (radical, cation) beforehand. For example, reactive radical particles may be formed after activation of particular compounds so-called initiators. The activation of the initiators may be carried out using photons, heating, oxidation-reduction potential or enzymatic activity. The initiator(s) have a radical centre and thus the ability to easily transfer the radicals to the reactive molecules. Thus, the radicals are formed in cascade. To sum up, once the initiator has been added and the stimulus applied, where appropriate, the radicals start to be generated and the polymerisation is initiated. During the process of growth of the polymer, a chain including monomer units is formed. Monomers are added gradually at the ends of the chains of the polymers. The polymerisation process may be interrupted, in particular when reaching a high conversion rate, a sudden recombination of several different chains with one another or of the presence of free radical inhibitor compounds in the reaction medium. Oxygen is typically used as a radical inhibitor. Consequently, it is preferable to proceed with a degassing before the polymerisation and/or apply inert conditions during the latter. This requires complex manipulations and involves many laboratory equipment, such as drying agents, inert gas tanks or an inert gas line, pumps, etc. Furthermore, the chain polymerisation generates the formation of chains of different lengths, which generates irregularities in the gel matrix. The gel matrices also contain heterogeneous transverse bonds with high molecular weight, which results in a hydrogel consisting of meshes of variable sizes. Thus, the structure of the gel is often incompatible with nucleic acid amplification reactions. On the other hand, radical polymerisations are generally rapid and consequently barely controllable, if not at all.

The step-by-step polymerisation involves different types of reactions of bifunctional or multifunctional monomers (pre-polymers). In particular, the thiol-based Michael-type conjugation reaction involving PEG derivatives is commonly used in this type of polymerisation. By definition, the functional groups of the monomers or pre-polymers are reactive sites and can thus react directly with one another. Unlike the chain polymerisation, dimers, trimers and tetramers are formed at the start of the polymerisation. Afterwards, these oligomers combine with one another to form long polymer chains. In general, the polymers formed by this type of polymerisation do not have any heterogeneous transverse bonds [Biomacromolecules 2012, 13 (8), 2410-2417; J. Appl. Polym. Sci. 2015, 132, 8]. This guarantees a certain homogeneity of the meshes. For this purpose, the matrices of gels of this type are preferred for PCR applications, such as the formation of polonies. Nevertheless, the structure of the gels is very compact, which is often incompatible with nucleic acid amplification reactions. In addition, the step-by-step polymerisation reaction could take place rapidly after mixing the reagents. It follows that some of these reactions are also barely, and even not at all, controllable.

Beyond the uncontrollable properties of the polymerisation reactions and the unsuitable structure of the gels, there are other drawbacks, some of which are discussed hereinbelow.

The reaction times of the known gelation processes such as those described hereinabove (acrylamide-bis-acrylamide copolymers and multi-arm PEG-acrylate/PEG-SH copolymers) often require between 10 and 30 minutes at room temperature or at temperatures comprised between 20 and 40°° C. to form an almost-complete polymer structure [P. Blainey et al. (U.S. Pat. No. 10,487,354); Huang et al. (US2019/0202268); G. M. Church et al. (U.S. Pat. No. 6,485,944); A. B. Chetverin et al. (EP1999268); A. B. Chetverin et al. (U.S. Pat. No. 6,001,568)]. This duration reduces the effectiveness of the amplification reagents which are generally sensitive to temperature (enzymes, fluorophores, etc.). Furthermore, this duration is unsuitable for all applications where the rapidity of the test is important, like, for example, so-called patient bedside applications (“Point-of-Care Testing” in English). For such applications, a gelation time period in the range of one minute would be desirable.

The start of a polymerisation reaction is uncontrollable in the techniques of the prior art. Indeed, in the case of PEG-acrylate-PEG-SH hydrogels with step-by-step growth, the reaction begins as soon as the components are mixed, and in the case of acrylamide-bis-acrylamide hydrogels with chain growth, the reaction begins upon mixing of the components with polymerisation initiators (for example APS-TEMED). This complicates the manipulations of the user (there is a need for speed in particular) and affects the repeatability of a protocol because of the high probability of formation of defects in the matrix of the gel during the process of mixing then injection into the reaction chamber.

In addition, it is impossible to automate the known polymerisation processes, since manipulation with replicas of 100-1,000 reactions and more requires the separate preparation of batches of 2-5 maximum reactions. This also complicates the use of amplification chambers of different shapes (cassettes, narrow or elongate reaction chambers, i.e. rod or capillary type ones, especially folded or wound in a flat cassette in order to be able to be placed in a standard PCR cycler), which limits their number to those which can be filled rapidly. More particularly, in known gel amplification methods, the viscosity of the reaction mixtures increases as soon as the polymers are introduced into the mixture. It follows that the reaction mixture rapidly gels and that it is necessary to introduce it rapidly into the amplification reaction chamber. Failing that, the gelled mixture could clog the inlet of the chamber. The mixture then becomes unusable and the amplification fails.

The chain radical reaction, for example in the case of acrylamide-bis-acrylamide gels, is a reaction sensitive to the presence of oxygen in the air. Oxygen inhibits the polymerisation and contributes to the formation of networks containing heterogeneous crosslinks with a high molecular weight. This results in hydrogels consisting of meshes of variable sizes. Consequently, it is preferable to perform a degassing process before the polymerisation reaction and/or to apply inert conditions during the latter. This requires complex manipulations and the use of several laboratory pieces of equipment, such as desiccators, inert gas tanks or inert gas pipes, pumps, etc.

In addition, a major drawback of these gels is that the unreacted acrylate monomers are harmful for the amplification reaction. It has been attempted to solve this problem by additional washing steps (A. B. Chetverin et al. in EP1999268 and U.S. Pat. No. 6,001,568). Yet, this increases the complexity of the processes by adding long steps, often associated with losses of yield. Indeed, the method for preparing the gels comprises: a) treating the surface of the glass with a binder-silane-water-ethanol-acetic acid mixture for fixing the gel (about 2 h); b) degassing the pre-polymer solution (at least about 15 min); c) incubating at 4°° C. for 1 h, then rapidly adding initiators and gelling at room temperature for about 40 min; d) washing the gel for about 60 min, followed by drying overnight at room temperature; e) diffusing the amplification reaction mixture into the gels. Thus, the entire process lasts about 13 h or more. Besides the time constraint, another major drawback of this type of process is that it makes the use of these gels almost impossible for target molecules of various sizes (DNA, RNA, bacteria, viruses). This is due in particular to a problem of diffusion within the gel which results in an uneven distribution of the amplification products.

In the context of nucleic acid amplification reactions, the preparation of gels should take account of a large number of factors. In particular, mention may be made of the physical stability of the gel, its inertia, the toxicity of the components, the compatibility of the gel with the amplification reaction reagents, as well as the compatibility of the gel with the amplification conditions (i.e. on the one hand the influence of the components of the gel on the amplification reagents and, on the other hand, the influence of the amplification reagents on the gelation).

It has been attempted to make PEG-based gels, in particular a copolymer of PEG 4-Arm acrylate and PEG 2-Arm thiol, and to apply therein a nucleic acid amplification reaction (P. Blainey et al. in U.S. Pat. No. 10,487,354 or Huang et al. in US2019/0202268). Nonetheless, the excessively rapid triggering of the polymerisation and its uncontrollable nature leads to difficulties which make industrialisation impossible.

In fine, the problems encountered in the art are diverse and various, and, to sum up, mention may be made of: a long preparation process; an initial polymerisation that is too rapid and non-controllable; an excessively long time for the formation of the gel; reproducibility problems; the formation of non-homogeneous gels; complex manipulations; degassing or manipulations under an inert atmosphere; protocols in a large number of steps; the need to wash the gels to eliminate potential inhibitors of the amplification of the nucleic acids; and the need for several laboratory pieces of equipment (desiccators, inert gas reservoirs or inert gas line, pumps, washing baths, etc.).

All this means that no solution of a nucleic acid amplification (in particular PCR) technique could be made in gel in a satisfactory manner. Moreover, to date, there is currently no gel amplification technique commercialised and/or industrialised.

The Applicant has developed a composition and a method for amplifying hydrogel nucleic acids which solves the problems of the art. Thus, the invention does not require complex manipulations, uses non-toxic reagents compatible with both the nucleic acids and the enzymatic amplification thereof. The polymerisation with the composition of the invention is controllable by means of a polymerisation agent which is sensitive to light and/or temperature. The polymerisation agent of the invention is photosensitive and/or heat-sensitive.

In particular, the invention offers (i) manipulation simplicity, (ii) speed, (iii) controllability, and (iv) a drastically increased storage in comparison with the prior state. Indeed, the invention allows (i.1) preparing the amplification reaction in one single step, and that being so without complex manipulations. In addition, (i.2) no degassing step is required (often related to an oxygen sensitivity). The (ii.1) preparation is rapid, i.e. in particular with no washing. For example, there is no need to wash the gels to eliminate any inhibitors of amplification enzymes. The (ii.2) gelation is very rapid, and that being so only after the application of a stimulus (luminous or thermal). The (iii.1) stimulus is space and time controllable, and is furthermore perfectly (iii.2) reproducible. The (iii.3) size of the polonies is homogeneous, which in particular allows further controlling the amplification process. The ingredients of the composition of the invention make (iv.1) mixing thereof possible without any risk of gelation before application of the stimulus. The (iv.2) long-term storage, as well as the completion of a large number of (iv.3) manipulations in parallel, become possible with the invention.

In particular, the invention relates to a polymerisation induced by light or by a threshold temperature of one or more monomer(s) or pre-polymer(s) with or without a photo-or thermo-initiation agent. The polymerisation of the invention combines the advantages of the above-described two polymerisation mechanisms (chain and step-by-step growth).

Photo-polymerisation and thermo-polymerisation are part of the chemical methods for forming hydrogels. It takes place by exposing a photosensitive or heat-sensitive system composed of unsaturated components with/without a photo-or thermo-initiator (with/without other components bearing photo-/heat-sensitive functional groups) to ultraviolet light (−200-400 nm) or visible light (−400-800 nm) or at a high temperature (higher than room temperature).

In the present description, and for the definition of the invention, by “stimulus” or “stimuli”, it should be understood any chemical or physical element or factor triggering a polymerisation.

The main advantage of a polymerisation activated by photons (photo-activation) or by heat (thermo-activation) is that the hydrogels are formed in situ from aqueous solutions in a minimally invasive manner and with control of the triggering of the gelation process. This is possible thanks to the use of an external stimulus applied to the reagents. The stimulus used in the invention are respectively the targeted irradiation of the pre-polymers with a light of selected wavelength or the exposure of the monomers (which are also pre-polymers) has a threshold temperature. The possibility of adjusting the intensity and the duration of exposure to light or the temperature by means of the PCR device allows for an additional control over the gelation and thus over the properties of the generated gel (control of the swelling rate, of the mechanical strength, of degradability, etc.).

As regards photo-activation: the light may be directed at a specific location and at a defined time point. This allows making different technical designs of the amplification chambers. The use of light energy enables polymerisation at low doses of initiator radicals under simple reaction conditions. Typically, the photo-polymerisation is defined by rapid polymerisation rates (about a few minutes) and a minimum heat production. This is particularly important for the stability of the amplification systems (Biomaterials 2002, 23, 4307-431). These advantages make this method of forming hydrogels particularly well suited to in vivo applications, to the encapsulation of viable cells for tissue engineering and regenerative medicine strategies, as well as to applications for the controlled release of medicines (B. Amsden. Chapter 8: Méthodes Chapter de photoréticulation pour concevoir des hydrogels. Handbook, pp. 201-218 (2016)).

As regards thermo-activation: the temperature range used during the PCR may be enough to initiate the polymerisation. Thus, the gelation could take place at the same time as the beginning of the nucleic acid amplification reaction. Unlike hydrogels formed by photo-polymerisation, this system could require no additional time related to irradiation with light. This makes this embodiment particularly suitable for rapid PCR applications. In addition, it does not require any additional equipment such as a UV lamp integrated into the amplification device (such as a PCR thermocycler, for example), since the stimulus of the formation of the hydrogel is the same as that one of the amplification of the nucleic acid, namely the temperature.

The invention provides for selecting one or more monomer(s) or pre-polymer(s) for the construction of a hydrogel. In general, the concentration of monomers or pre-polymers in the composition of the invention is comprised between 2% and 25%. More generally, the invention provides for a polymerisable reagent which polymerises under the effect of an external factor. According to the invention, this factor is a light or a temperature. Thus, when the polymerisable reagent (i.e. the monomers or the pre-polymers) is exposed to light (when it is photosensitive) or to the temperature (when it is heat-sensitive), the latter polymerises. A hydrogel is then formed.

Optionally, the composition of the invention may comprise a photo-initiator or a thermo-initiator. When a photo-initiator or a thermo-initiator is used, its amount is generally selected between 0.02% and 1%.

It should be noted that the present invention relates to a masterbatch composition intended to be mixed with a biological sample. This sample may include, or not, one or more target sequence(s) which are to be identified by amplification, in particular to identify the presence or absence of a pathogen in the sample.

Hence, the masterbatch composition includes ingredients necessary for the amplification of the target sequences. Thus, the composition includes in particular amplification reagents (for example dNTP's nucleic bases, MgCl₂, reaction buffer) and amplification enzymes (for example Taq polymerase, reverse transcriptase).

Hence, the composition does not include ab initio the target sequence or sequences to be amplified. Nor does the composition necessarily include the primers specific to the target sequence(s), or the fluorescent probes for revealing an amplification of the target nucleic acid sequence(s), since these two elements depend on the target sequence(s) that are sought. Logically, when the desired target sequence is known (for example a coronavirus-specific sequence), the composition may contain the primers and probes specific to this sequence.

Nonetheless, in the present description, reference is generally made to the composition of the invention in its state already mixed with the target sequence (including the primers and the probes). The technical effect related to the polymerisation of the polymerisable reagent of the invention is observed under the conditions of use of a method for amplifying nucleic acids.

Indeed, conventionally, the composition further includes enzymes for the amplification of nucleic acids, primers that are more or less specific to the target sequence(s), one or more fluorescent marker molecule(s) more or less specific to the target(s) of interest, a reaction buffer and other additives, where necessary.

The composition of the invention further includes, in the use state, a sample to be analysed (DNA, RNA, plasmid, bacteria, viruses, phages, fungi, etc.) including target nucleic acid sequences. It should be noted that the sample may initially be in a second mixture, and/or undergo a prior purification in particular. In this case, the sample is mixed with the constituents of the gel and the PCR reagents. The resulting mixture is a composition according to the invention.

The composition may be injected into a reaction chamber compatible with a nucleic acid amplification instrument. Thus, the mixture is in a liquid state (more or less viscous) inside the reaction chamber and could thus be adapted to different shapes of reaction chambers.

According to the invention, the hydrogel formed by the polymerisation of the polymerisable reagent has the effect of locally encapsulating each target nucleic acid sequence, while preserving a diffusion of the other constituents of the composition to enable an effective amplification of the target nucleic acid sequence(s).

When the composition of the invention is in the gel state in the reaction chamber(s), the target nucleic acid(s) amplification process could be carried out. In particular, the amplification process may be selected from among one of those mentioned hereinabove in the description, in particular a conventional or qPCR-type PCR, real-time PCR, asymmetric PCR, RT PCR, or others; a NASBA; a 3SR (“Self-Sustained Sequence Replication” in English); an SDA; a TMA; an RCA; a LAMP, or an MDA (“multiple displacement amplification” in English).

FIG. 1 shows a block diagram of the method of the invention. The amplification reaction mixture (Mix PCR) and a polymerisable reagent (polymers+activator(s)) are mixed to form the masterbatch of the invention (MasterMix Polonies). Thus, the masterbatch is ready for use and is mixed with the biological sample (target) containing the target sequence(s).

Thus, the composition of the invention including the target sequences and the primers, in particular, could be distributed in reaction chambers of a nucleic acid amplification device (amplification chamber(s)). Afterwards, a factor selected from among a light or a temperature is applied so as to initiate the polymerisation of the polymerisable reagent. Thus, a hydrogel according to the invention is formed. According to the invention, the hydrogel locally encapsulates each target nucleic acid sequence while enabling the diffusion of the amplification reagents. The amplification process could then be carried out (Amplification).

The Applicant has discovered, not without surprise, that the selection of the polymers, and of the activators where appropriate, conditions the state of the formed hydrogels. The hydrogels formed from the composition of the invention have characteristics which solve the problems mentioned hereinbefore in the description.

To select the polymers and activators of the invention and thus form a hydrogel from the composition of the invention, it should be proceeded as follows.

The first step includes the design of the hydrogel matrix according to the final needs. The main considerations to be taken into account are: a) the chemical origin of the components of the hydrogel, their compatibility with the protocol; b) the use of functionalised monomers or pre-polymers; c) the type of photo-crosslinking or thermos-crosslinking reaction; d) optionally the type of photo-crosslinking or thermos-initiator, as well as the source to be applied, i.e., respectively, the light source or the heat source; e) the structure and the density of the desired network (percentage of gel, ratio of the reagents).

Various natural and/or synthetic materials may be used to form photo-or thermo-crosslinkable hydrogels. Natural substances, such as alginate, hyaluronic acid, chitosan, collagen, silk fibroin and gelatin may be applied. However, it should be noted that the variation, from one batch to another, of the hydrogels of natural origin does not always reproduce their mechanical and biochemical properties and, consequently, could limit the possibility of obtaining matrices with well-defined properties. The material properties of the synthetic hydrogels may be adjusted accurately. To the extent that these materials are free of biologically relevant functions, some applications require the introduction of specific characteristics present in the natural extracellular matrices (ECM) in a highly controlled manner. Synthetic materials may include, without being limited thereto, Pluronics derivatives, polyvinyl alcohols (PVA), poly (acrylic acids), polyethylene glycols (PEG) and polypeptides (Chinese Chem. Lett. 2020; ACS Macro Lett. 2013, 2, 5-9).

The use of monomers in the polymerisation is limited by the fact that most of them are cytotoxic and/or carcinogenic, and have inhibitory effects on the amplification process. In addition, this approach often gives rise to inhomogeneous networks. An alternative solution consists in using macromolecular hydrogel precursors (pre-polymers) which are functionalised with photo-or thermo-reactive groups for the formation of photo-or thermo-polymerisable hydrogels respectively. Two advantages of the pre-polymers are their low toxicity on the one hand and their low inhibition rate on the other hand, in comparison with the acrylamide/bis-acrylamide system, for example. In addition, the pre-polymers are stable and water-soluble. As discussed hereinabove, these molecules have at least one, and possible several, reactive functional group(s). For example, it is possible in particular to use PEG acrylate and methacrylate derivatives (linear and multi-arm configurations), PEG norbornene derivatives (linear and multi-arm configurations), PEG thiol derivatives (linear and multi-arm configurations), PEG acrylamide and methacrylamide derivatives (linear and multi-arm configurations), polyvinyl alcohol (PVA) derivatives, modified polysaccharides such as hyaluronic acid derivatives, dextran methacrylate. The peptides may be incorporated into the PEG-acrylate hydrogels by functionalisation of the terminal amino groups of the peptide with an acrylate portion (Biomaterials 2002, 23, 4307-4314).

There are several types of photo-crosslinking or thermo-crosslinking reactions. The selection of the appropriate reaction is made according to the amplification reaction and the applied targets, and the temperature range in particular. Preferably, in the context of the present invention, a bio-orthogonal thiol-ene reaction and a dimerisation reaction are used.

As regards the photosensitive polymerisation, among the bio-orthogonal reactions, the photo-click polymerisation of thiol-norbornene (thiol-ene) is one amongst the options preferred here. This polymerisation proceeds by a step-by-step growth mechanism, enabling the manufacture of hydrogels structurally uniform with almost no network defect. It involves orthogonal reactions mediated by light between multifunctional macromers containing norbornene and thiol functions at the ends. Activated by UV or visible light, the thiolene reaction is reflected by the rapid addition, step-by-step, of thiols to the double bonds of the norbornene fragments, with the formation of thioether bonds, and with a radical mediation (it should be noted that a cationic ionic mechanism is also possible). This type of bio-orthogonal reactions is insensitive to water and oxygen, does not require the addition of a co-initiator or a co-monomer, and could take place under mild, selective and efficient reaction conditions. This results in the formation of a homogeneous network and rapid kinetics in comparison with the chain polymerisation. The mechanism of the step-by-step polymerisation allows obtaining a homogeneous network with the appropriate meshes which drastically limit the diffusion of the target molecules/cells and of the amplification products, but enable the diffusion of the amplification reagents.

Another approach in the context of photosensitive polymerisation is the application of cycloaddition reactions, for example photodimerisation. The photodimerisation takes place between two same unsaturated molecules (after activation by light) and results in the formation of a dimer. Different structures, containing double bonds such as coumarins, cinnamates, thymine, anthracenes, stilbene, chalcones and dimethylmaleimide (DMMI) may be used. They may be integrated into natural or synthetic polymers, such as: cinnamate, coumarin and thymine conjugates with hyaluronic acid or chondroitin sulphate; cinnamate conjugates with poly (N, N-dimethylacrylamide), PEGs containing terminal cinnamate groups and nitrocinnamate groups (Adv. Funct. Mater. 2001, 11, 1); gelatin modified with nitrocinnamate; PVA with coumarin groups; poly (acrylamide) bearing DMMI. (Chapter 8: Photo-Crosslinking Methods to Design Hydrogels. Gels Handbook. Gels Handbook, pp. 201-218 (2016)). The photoreactivity of these compounds could be adjusted by varying the substituents of the photosensitive reactive centre. Among the additional advantages of this crosslinking strategy, it should be noted that no initiator or catalyst is necessary. On the other hand, a high energy supply should be applied to obtain an effective crosslinking. The UV intensity and the gelation time may be reduced in several ways. The first one consists in incorporating a larger number of photodimerisable groups on the core of the pre-polymer, which will also increase crosslinking (for example, by incorporating photodimerisable fragments over the entire length of the skeleton of the pre-polymer, using multichain pre-polymers). The second one assumes the use of water-soluble sensitisers, such as thioxanthone disulphate, to reduce the energy supply (Polymer 2007, 48, 5599-5611).

Another group of photo-induced polymerisations comprises reactions with the formation of intermediate reactive nitrene radicals from azides activated by UV light. The nitrene radical could rapidly be subjected to addition reactions with unsaturated bonds, insertion reactions in carbon-hydrogen or nitrogen-hydrogen bonds. Hydrogels may also be formed when the nitrene radical reacts with primary amines, for example modified chitosan (B. Amsden. Chapter 8: Photo-Crosslinking Methods to Design Hydrogels. Gels Handbook. Gels Handbook, p. 201-218 (2016)).

The selection of the suitable photo-initiator (where necessary) is a crucial task in the design of photo-crosslinkable matrices for the amplification of nucleic acids. A series of important characteristics should be taken into account: a photo-initiator should have an absorption spectrum which has a good overlap with the emission spectrum of the desired light source; a rather high value of the molar extinction coefficient; good solubility in water; stability; capacity to produce free radicals (or other reactive particles); biocompatibility, compatibility with the amplification process, etc. (Biomaterials 2002, 23, 4307-4314; BioTechniques 2019, 66, 40-53).

As regards the heat-sensitive polymerisation, among the bio-orthogonal reactions, the Diels-Alder reaction is one of the preferred options here. It proceeds with a step-by-step growth mechanism, enabling the construction of structurally uniform hydrogels with almost no defects. It involves orthogonal reactions mediated by the temperature between multifunctional macromers containing diene and dienophile functions. Different structures may be used for the conjugated diene (furan, tetrazine, etc.) and for the substituted alkene (maleimide, norbornene, etc.). These structures may be integrated into a natural or synthetic polymer such as poly (acrylates) (European Polymer Journal, 2013, 12, 3998-4007), poly (etheramines) (Polymers, 2019, 11, 930), hyaluronic acid (Polymer Chemistry, 2014, 5, 5116-5123), alginate (Biomaterials, 2015, 50, 30-37). Activated by temperature, the Diels-Alder reaction is a radical-free reaction which results in a six-membered ring. This type of bio-orthogonal reactions is insensitive to water and oxygen, does not require the addition of a co-initiator or a co-monomer, and could take place under mild, selective and efficient reaction conditions. This results in the formation of a homogeneous network and fast kinetics compared to chain polymerisation. The mechanism of the step-by-step polymerisation allows obtaining a homogeneous network with the appropriate meshes which drastically limit the diffusion of the target molecules/cells and of the amplification products, but enable the diffusion of the amplification reagents.

Another approach in the context of heat-sensitive polymerisation is the dimerisation of a polymerisable group such as acrylate. Dimerisation takes place between two of the same polymerisable functions in the presence of a thermo-initiator. Different structures containing double bonds such as (meth) acrylates, (meth) acrylamides or maleimides may be used and these structures may be integrated into natural or synthetic polymers, such as pegs or polysaccharides (hyaluronic acid, gelatin, etc.). The thermoreactivity of these compounds may be adjusted by varying the substituents of the heat-sensitive core. The temperature and the gelation time may be reduced in several manners. The first consists in incorporating a larger number of thermodimerisable groups on the core of the pre-polymer, which will also increase crosslinking (for example, by incorporating thermodimerisable portions along the entire skeleton of the pre-polymer, using multi-arm pre-polymers). The second one assumes the use of higher concentrations of thermo-initiator, while paying attention to compatibility with PCR.

The selection of a suitable thermo-initiator (where necessary) is a crucial task in the design of thermos-crosslinkable matrices for the nucleic acid amplification. A series of important characteristics should be taken into account: a thermo-initiator should have a decomposition temperature which has a good overlap with the temperature used during the PCR process; a rather high value of the molar extinction coefficient; good solubility in water; stability; ability to produce free radicals (or other reactive particles); biocompatibility, compatibility with the amplification process in particular. Different types of thermo-initiators may be used to initiate a thermal polymerisation: —Persulphates, neutralised with a counterion (ammoni um, potassium, sodium, etc.), decompose into sulphate radicals which, in an aqueous solution, form HSO⁴⁻ ions and hydroxyl radicals, capable of initiating the polymerisation (Catalysis Today, 2015, 257, 297-304); —Organic peroxides (methyl ethyl ketone peroxide, benzoyl peroxide, etc.) which undergo symmetrical fission (homolysis), forming two radicals capable of initiating the polymerisation; —Azo compounds (2,2′-azobis (isobutyronitrile), 2,2′-azobis [2-(2-imidazolin-2-yl) propane dihydrochloride], etc.) which decompose with heat (or light) thereby forming gaseous nitrogen and carbon radicals capable of initiating the polymerisation.

The polymerisation process takes place by the formation and the reaction of some reactive species, i.e. radicals or ions. They may be formed as a result of a high chemical reactivity of the participating components themselves (for example cations in a Michael-type addition); following a special activation by one or more initiator(s) (for example, radicals formed following an oxy-reduction reaction of the APS-TEMED system in a polymerisation chain), by application of temperature with or without initiators (for example, ammonium persulphate (ammonium persulphate, APS in English) which could be decomposed into radicals by heating) or by application of light with or without a photo-initiator.

In the present invention, this last approach is used, i.e. the application of light or temperature respectively with or without a photo-initiator or a thermo-initiator for the generation of reactive radical species. In general, two main groups of initiators are used in the synthesis of hydrogels for biomedical applications: radical and cationic photo-initiators or thermo-initiators (i.e. those which induce the formation of reactive radicals or cations in the system, respectively). The use of cationic photo-initiators or thermo-initiators is possible but complicated because they lead to the formation of protonic acids, which are harmful to the cells and the enzymes, which could inhibit the amplification process.

In the context of photosensitive polymerisation, cleavable photo-initiators (type I) or biomolecular photo-initiators (type II) may be provided. Type I photo-initiators include, without limitation, benzoin and acetophenone derivatives, such as the group of Irgacure photo-initiators (Irgacure-2959; Irgacure-184; Irgacure-651; Irgacure-369; Irgacure-907; etc.), lithium phenyl-2, 4, 6-trimethylbenzoylphosphinate (LAP), etc. Under the effect of light, these compounds decompose into two portions bearing radical centres, capable of promoting the subsequent polymerisation. Typically, this type of compound has a maximum in the absorption spectrum around 300-400 nm and, consequently, can be used for UV polymerisation. On the contrary, type II photo-initiators, such as camphorquinone, thioxanthone, benzophenone, have a maximum in the absorption spectrum in the visible range and can be used for polymerisation mediated by visible light. The mechanism of their action is based on the extraction of the hydrogen present in the co-initiator (for example, thriethanolamine) with the generation of secondary radicals. Often, to induce an efficient photo-gelation, an accelerator, such as N-vinylpyrrolidone, is necessary. However, compared with type I photo-initiators, type II is less cytotoxic and more soluble in water. One of the photo-initiators sensitive to the most widely used UV light is Irgacure-2959, which is due to its moderate solubility in water, its low cytotoxicity and its minimum immunogenicity. However, it is characterised by an initiation efficiency and a molar extinction coefficient in the UV-A spectral range that are relatively low. A good alternative consists in using LAP, which is more efficient and biocompatible, and which, being a lithium salt, is well soluble in water. In addition, LAP also absorbs in the region of blue light (405 nm). It is also possible to implement V-50 (2,2′-azobis (2-methylpropionamidine) dihydrochloride) and V-086 (2,2′-azobis (N-(2-hydroxyethyl)-2-methylpropionamide)) (BioTechniques 2019, 66, 40-53; B. Amsden. Chapter 8: Photo-Crosslinking Methods to Design Hydrogels. Gels Handbook, p. 201-218 (2016)).

The optimum concentration of the photo-initiator or of the thermo-initiator is an important parameter for the polymerisation of the hydrogels used in the amplification process. High concentrations of the initiator enable the generation of more free radicals, which normally results in a higher conversion of the monomers. However, an excessively high concentration of the initiator could be harmful for amplification because the radicals could damage the cellular macromolecules, such as cell membranes, proteins and nucleic acids, but also fluorophores. This results in an inhibition of the amplification. Preferably, a concentration of 0.02%-1% is used depending on the applied initiator, the intensity of the light or of the temperature, the mechano-kinetic aspects of the polymerisation.

Finally, the variation of the percentage of gel and of the ratio of the components allows obtaining the desired structure and network density. The gel matrix should have a homogeneous structure with the appropriate meshes which limit the diffusion of the target molecules/cells and of the amplification products, while enabling the diffusion of the amplification reagents. An average pore size ranging from 100 μm to 5 nm is capable of preventing the diffusion and thus the intermixing of the target sequences, while enabling the diffusion of the amplification reagents. In this range, the concentration of the gel may be modified in order to influence two parameters: a) the size of the molecular colony or polony; b) the restriction capacity of the matrix of the gel against different types of targets (DNA, RNA, plasmids, bacteria, viruses, phages, etc.). As pointed out before (G. M. Church et al. U.S. Pat. No. 6,485,944; A. B. Chetverin et al. EP1999268, A. B. Chetverin et al. U.S. Pat. No. 6,001,568), the increase in the percentage of gel results in a reduction in the size of the molecular colonies. The concentration of the gel also has a considerable influence on the capacity of the matrix to trap different targets.

Different reaction chambers may be used. They may include, without being limited thereto, flat chips, cassettes, narrow or elongated reaction chambers, i.e. of the rod or capillary type, especially folded or wound in a flat cassette to enable placement in a standard PCR thermocycler. The chamber may be of different suitable volumes confined in a closed space excluding the inlet/outlet holes for pouring the mixture.

The reaction chamber consists of different portions. In a particular embodiment of the invention, the lower portion of this chamber is in direct contact with a heating system on one side, and with the mixture (liquid or gel) on the other side. Preferably, it is flat to ensure the better adhesion of the gel matrix. The upper portion, the visible portion, is also preferably planar, and is tightly bonded to the base portion. The upper portion should be transparent both to the light wavelengths in order to be able to initiate the polymerisation and at the light wavelengths of the amplification reaction detection process. The distance between the base and the top may be comprised between 1 μm and 1 mm, preferably that one which will enable the arrangement of a monolayer of the amplification products, i.e. molecular colonies. The other two dimensions (the length and the width) should be larger (ideally with a minimum factor of 10) in order to provide a sufficient surface area for the placement of different targets and amplification products thereof. To avoid dehydration of the gel, the inlet/outlet holes should be closed in different manners: for example, mechanically, which is integrated by the design of the chamber; with the use of external adhesive films, and/or with the use of pressure.

According to the first embodiment of the invention, the composition is exposed to light with selected wavelength and intensity for a predefined time period. A light of different wavelengths (UV at 200-400 nm or visible light at 400-800 nm) and of different intensities may be applied. The irradiation intensity and dose should necessarily be taken into account, since they are necessary for initiating the polymerisation and for increasing the photoinitiation. It should be noted that there is no linear proportionality between the intensity of UV light and the photoinitiation and that excessively high intensity values could lead to a degradation of the resulting polymer into an inhomogeneous gel. In addition, an excessively high intensity could cause the destruction of reagents necessary for amplification. The range of UV intensities between 1 and 20 mW/cm² is adapted and allows forming a gel in 1-5 minutes.

According to the second embodiment of the invention, the composition is exposed to a threshold temperature. In particular, it may be sufficient to begin the PCR to initiate the polymerisation of the composition of the invention.

When the gel is formed, the nucleic acid sequence or sequences are immobilised in the matrix of the gel. The amplification could then be carried out on an amplification device. Mention may be made of different machines, for example the MasterCycler Nexus flat thermocycler from the Eppendorf company; the PCR ProFlex system from the Applied Biosystems company; the QuantStudio 3D from the Applied Biosystems company; SmartCycler from the Cepheid company, or Chronos by the Applicant.

### EMBODIMENTS

Examples 1 to 5 relate to hydrogels obtained from compositions including a photosensitive polymerisable reagent according to the invention, and Examples 6 to 8 relate to hydrogels obtained from compositions including a heat-sensitive polymerisable reagent according to the invention. Unless stated otherwise, the PCRs are carried out on a commercially-available Chronos machine.

### EXAMPLE 1

### Bare DNA in Symmetrical PCR

A bio-orthogonal thiol-ene reaction is used for the formation of the hydrogel. The equimolar amounts of 4 Arm-PEG-SH (MW of 10,000 g/mol, Laysan Bio) and 4 Arm-PEG-norbornene (MW of 10,000 g/mol, Sigma) are selected for the gel construction with percentages (%) w/v in the range of 5-7%. LAP (lithium phenyl-2, 4, 6-trimethylbenzoylphosphinate, Sigma) is used as a photo-initiator at a rate of 0.02% (w/V). The following amplification reagents are used: 1 U of SsoAdvanced Universal SYBR Green Supermix (Bio-Rad), 8U of Platinum™ Taq DNA polymerase (ThermoFisher SCIENTIFIC), 0.4 μM of primers targeting the 16S region of the bacteria with a matrix length of 740 pb. Optionally, an additional intercalating dye (SybrGreen® fluorescent DNA dye (Jena Bioscience) or EvaGreen® dye (20X in water, Biotium) may be added at concentrations in a range of 0.5-1.5 μM).

The mixture containing all of the components with the target DNA originating from B. Subtilis is adjusted with ddH₂O at 25 μL and injected into the reaction chamber. Depending on the used thermocycler, the chamber is characterised by the following dimensions: SmartCycler from Cepheid (chamber made of plastic of 5×5×1 mm, 25 μL); the flat thermocycler MasterCycler Nexus from Eppendorf (chamber of 9×9×0.3 mm, 25 μL (silane-treated glass microscope slide+frame sealed chamber (Bio-Rad)+cover slip made of plastic)); Chronos (chamber of 10×10×0.25 mm, 25 μL with a base made of Al and a lid made of cyclic olefin copolymer (COP)). In this case, the SmartCycler machine from Cepheid is used.

The gel resulting from the photosensitive composition has been formed at room temperature after 1 minute in a thiol-norbornene reaction activated by the application of UV light (365 nm, 3.4 mW/cm²) generated by a light source.

The PCR is carried out through 2 min at 95°° C. for the initial denaturation, then 35 cycles of 5 s at 95° C. for the denaturation and 30 s at 60° C. for the elongation.

The B. Subtilis DNA molecular colonies (104, 103, 102 copies and a negative control) are visualised with the Axiovert 100 (ZEISS, Germany) fluorescence microscope.

FIG. 2 shows the respective photographs of the hydrogel of the samples including 104, 103, 102 copies and a negative control after PCR. FIG. 2 also shows a curve of the fluorescence signal as a function of the PCR cycles and the respective Ct values.

The photos are captured with a Samsung Galaxy S 10. The negative control camera containing 20-30 molecular colonies due to the amplification of the impurities of E. Coli present in the SsoAdvanced Universal SYBR Green Supermix, amplifiable with very sensitive primers targeting the 16S region.

### EXAMPLE 2

### Bare DNA in Symmetrical PCR

In this example, a bio-orthogonal thiol-ene reaction is used for the formation of the polymer matrix. More particularly, the equimolar amounts of 2 Arm-PEG-SH (MW of 3,400 g/mol, Laysan Bio) and 8 Arm-PEG-norbornene (tripentaerythritol) (MW=20,000, Jenkem Technology) are considered for the construction of the gels with percentages (%) w/V in the range of 6-11%.

LAP (lithium phenyl-2, 4, 6-trimethylbenzoylphosphinate, Sigma) is used as a photo-initiator in an amount of 0.02% (w/V). The same amplification reagents as those of Example 1 are used.

The composition, including the target DNA sequence originating from B. Subtilis (10³ copies), is adjusted with ddH₂O at 25 μL and injected into the reaction chamber.

The polymerisation of the composition is initiated by application of UV light (365 nm, 3.4 mW/cm²) originating from a UV torch. The gel is formed (at room temperature) after 1 minute by a step-by-step thiol-norbornene polymerisation reaction.

FIG. 3 shows that the number and the size of the points (which correspond to the polonies) were different according to the percentage of gel: 80-100 μm for a gel at 10.8% (photograph to the left), 100-120 μm for a gel at 8, 8% (photograph at the middle) and 150-160 μm for a 6% gel (photograph to the right). The increase in the concentration of the gel results in the formation of fewer and smaller points. The stronger signals for the gel at 6% and 8.8% could be explained by a higher efficiency of the PCR which is due to the larger size of the meshes of the matrix of these gels in comparison with the mesh of the gel at 10.8%. A larger mesh leads to a more free diffusion of the amplification reagents and in particular of relatively large molecules such as enzymes. On the other hand, a larger mesh also leads to an increased diffusion of the PCR products, which is at the origin of larger points. The PCR is carried out on a Cepheid SmartCycler device according to the protocol of Example 1 and the photographs are captured with a Samsung Galaxy S 10 camera on an Axiovert 100 (ZEISS, Germany) fluorescence microscope.

FIG. 3 also shows a curve of the fluorescence signal as a function of the PCR cycles and the respective Ct values.

FIG. 3 also shows a curve of the derivative of the fluorescence signal with respect to temperature, as a function of the temperature. The observed peak corresponds to the melting point of the product formed by amplification. In this case, a value of about 88°° C. is obtained, which value is expected for the amplified DNA.

### EXAMPLE 3

### Bare DNA in Symmetrical PCR

In this example, a dimerisation reaction is

used for the formation of the polymer matrix: a nitrocinnamate-based 8 Arm-PEG (MW of 21.400 g/mol) is used for the construction of the gels with a percentage (%) w/V of 10%. The following amplification reagents are used: 50 of SD Polymerase Hotstart (Bioron), a buffer (SD Polymerase Reaction Buffer incomplete (10x), Bioron), a 3 mM MgCl₂ buffer (Bioron), 0.4 μM of primers targeting the 16S region of the bacterium with a matrix length of 740 pb. A DNA intercalating dye EvaGreen® Dye, 20X in Water (Biotium) is added at a concentration of 2X. The composition containing all of the components, including the bare DNA target originating from E. Coli, is adjusted with ddH₂O at 25 μL and injected into the Chronos reaction chamber.

The gel resulting from the photosensitive composition is formed at room temperature after 1 min by a dimerisation reaction activated by the application of UV light (365 nm, 3.4 mW/cm²) generated by the UV torch. The PCR amplification process is carried out according to the following protocol: 2 min at 95° C. for initial denaturation, 35 cycles of 5 s at 98° C. for the denaturation and 30 s at 60° C. for the elongation. The molecular colonies are visualised using the Axiovert 100 (ZEISS, Germany) fluorescence microscope.

FIG. 4 shows nine photographs of hydrogels formed by different embodiments of the composition according to the invention having an identical initial concentration of target sequences (10³ copies) or zero (cf. the control images with negative samples), corresponding to the PCR data of some embodiments.

The first line of FIG. 4 shows a first photo captured with a Hamamatsu camera (10³ copies) and a second photo captured with the Apple iphone 11 camera (negative). The negative control contains a few molecular colonies due to the presence of impurities amplified by the very sensitive primers targeting the 16S region.

### EXAMPLE 4

### Bare DNA in Asymmetric PCR

In this example, a bio-orthogonal thiol-ene reaction is used for the formation of the polymer matrix: equimolar amounts of 4 Arm-PEG-SH (MW of 10,000 g/mol, Laysan Bio) and 4 Arm-PEG-norbornene (MW of 10,000 g/mol, Sigma) are considered for the construction of the gels with a percentage (%) w/V of 6.4%. LAP (lithium phenyl-2, 4, 6-trimethylbenzoylphosphinate, Sigma) is used as a photo-initiator in an amount of 0.02% (w/V). The following amplification reagents have been used: 50 from SD Polymerase Hotstart (Bioron), a buffer (SD Polymerase Reaction Buffer incomplete (10x), Bioron), 3 mM MgCl₂ (Bioron), a pair of tailored primers targeting the FimH gene of E. Coli with a matrix length of 400 pb. An EvaGreen® Dye, 20X intercalation dye in water (Biotium) has been added at the concentration of 1X.

The composition comprising a target sequence (104 copies of bare DNA) of E. Coli is adjusted with ddH₂O at 25 μL and injected into the Chronos reaction chamber.

The gel resulting from the photosensitive composition has formed at room temperature after one minute in a thiol-norbornene click reaction with a step-by-step growth activated by application of UV light (365 nm, 3.4 mW/cm²) generated by the UV torch.

The PCR amplification protocol is: 2 min at 92° C. for initial denaturation, 40 cycles of 5 s at 92° C. for the denaturation and 30 s at 64° C. for the elongation. The molecular colonies are visualised in real-time with the Chronos camera.

### EXAMPLE 5

### Virus in RT-PCR

The experimental protocol is that one used in Example 1, except that the reverse transcriptase iScript™ (Bio-Rad) is used for reverse transcription. In addition, the amplification protocol comprises a reverse transcription step for 60 s at 60° C. A pair of tailored primers targeting MS2 with a length of 200 pb is used. The networks of molecular colonies of MS2 (10³ copies) are visualised on the Chronos machine.

The third line of FIG. 4 shows a photo captured with the camera of the Chronos machine (10³ copies).

### EXAMPLE 6

### Bare DNA in PCR

This example implements an acrylate dimerisation reaction for the formation of the polymer matrix: 4 Arm-PEG-acrylate (MW of 20,000 g/mol, Laysan Bio) is considered for the construction of the gels at 10% w/V The VA-044 (2,2′-Azobis [2-(2-imidazolin-2-yl) propane dihydrochloride], TCI Chemicals) is used as a thermo-initiator in an amount of 0.07% (w/V). The following amplification reagents are used: 60 SD Polymerase HotStart (Bioron), a 10X buffer containing 250 mM of potassium acetate (KOAc), 83 mM of ammonium sulphate ((NH₄) 2SO₄), 30 mM of magnesium chloride (MgCl₂), 1.5 M of Tris-HCl and 1 V % of Twee-20, 0.2 μM of dNTP's mix (Sigma) and 0.4 μM of primers targeting the 16S region of the bacteria with a matrix length of 740 pb. Optionally, an additional intermediate dye (SybrGreen® fluorescent DNA dye (Jena Bioscience) or EvaGreen® dye (20X in water, Biotium) may be added at concentrations in a range of 0.5-1.5 μM).

The mixture containing all of the components with the target DNA originating from B. Subtilis is adjusted with ddH₂O at 25 μL and injected into the reaction chamber. Depending on the used thermocycler, the chamber is characterised by the following dimensions: SmartCycler from Cepheid (chamber made of plastic of 5×5×1 mm, 25 L); flat thermocycler MasterCycler Nexus from Eppendorf (chamber of 9×9×0.3 mm, 25 μL (silane-treated glass microscope slide+frame sealed chamber (Bio-Rad)+cover slip made of plastic)); Chronos (chamber of 10×10×0.25 mm, 25 μL with a base made of Al and a lid made of cyclic olefin copolymer (COP)). The Chronos machine is used here.

The gel formed by the heat-sensitive composition forms during the PCR thanks to the cycles between the denaturation temperature and the elongation temperature. Indeed, the threshold temperature enables the thermo-initiator to polymerise via the acrylic functions of the 4 arm-PEG.

The PCR is carried out through 2 min at 95° C. for the initial denaturation, then 35 cycles of 5 s at 95°° C. for the denaturation and 30 s at 60° C. for the elongation.

The B. Subtilis DNA molecular colonies are visualised with the Axiovert 100 (ZEISS, Germany) fluorescence microscope and the photos captured with the Apple iphone 11 camera.

The fourth line of FIG. 4 shows two photos captured with Apple iphone 11 (10³ copies and negative control). The negative control contains a few molecular colonies due to the presence of impurities amplified by the very sensitive primers targeting the 16S region.

### EXAMPLE 7

### Bare RNA in RT PCR

The experimental protocol is that one used in Example 6, except that the reverse transcriptase WarmStart® LAMP (New England Biolabs) is used for reverse transcription, and that the amplification protocol further comprises the step of reverse transcription for 60 s at 60° C. A pair of tailored primers targeting MS2 with a matrix length of 200 pb instead of that one used before to target the 16S gene of B. Subtilis. The molecular colonies of the bare RNA MS2 are visualised with the camera of the Chronos machine. The fifth line of FIG. 4 shows two photos captured with the camera of the Chronos machine (10³ copies and negative control). The negative control contains a few molecular colonies due to the presence of impurities amplified by the very sensitive primers targeting the 16S region.

### EXAMPLE 8

### Virus in RT-PCR

The protocol is that one used in Example 6, except that the reverse transcriptase WarmStart® LAMP (New England Biolabs) is used for reverse transcription, and that the amplification protocol further comprises the step of reverse transcription for 60 s at 60° C. A pair of tailored primers targeting MS2 with a matrix length of 200 pb is used. The molecular colonies of the bacteriophage MS2 are visualised with the camera of the Chronos machine.

The sixth line of FIG. 4 shows a photo captured with the camera of the Chronos machine (10³ copies).

In view of the embodiments related to photosensitive polymerisation, the Applicant has developed a device especially suited for the use of the composition of the invention. Thus, the present description further discloses an invention relating to a device for amplifying nucleic acid sequences. This device may be defined as follows:

A device for amplifying nucleic acid sequences comprising a thermocycler arranged so as to carry out a series of thermal cycles with a reaction mixture for amplifying nucleic acids including one or more target nucleic acid sequence(s), and amplification reagents comprising specific primers of the target nucleic acid sequence(s), fluorescent probes for revealing the amplification of said target sequences, and nucleic acid amplification enzymes, the reaction mixture further comprising a photosensitive reagent, polymerisable when exposed to light originating from the light source so as to form a gel; the device further comprising a reaction chamber arranged in the thermocycler and arranged so as to receive the nucleic acid amplification reaction mixture, a light source directed towards said reaction chamber, and a control of the light source so as to switch it on or switch it off and control the wavelength and the intensity of the light.

Hence, the device is arranged so as to generate a stimuli to the photosensitive reagent so that said reagent polymerises when it is exposed to the light originating from the light source. This results in the formation of the gel.

In other words, when the light source illuminates the reaction mixture, a hydrogel is formed locally encapsulating each target nucleic acid sequence and the amplification reagents.

The invention may also be commercialised in the form of a kit including the composition as described hereinbefore and a device for amplifying nucleic acid sequences. Thus, the invention also relates to: a kit for amplifying nucleic acid sequences including (i) the composition according to the invention and (ii) a device for amplifying nucleic acid sequences comprising a thermocycler arranged so as to carry out a series of thermal cycles with said composition, a reaction chamber arranged in the thermocycler and arranged so as to receive the nucleic acid amplification reaction mixture, and further comprising a light source directed towards said reaction chamber as well as a control of said light source so as to switch it on or switch it off and control the wavelength of the light.

1. A masterbatch composition for the amplification of nucleic acid sequences, intended to be mixed with a biological sample containing one or more target nucleic acid sequence(s) and specific primers of the target nucleic acid sequence(s), as well as fluorescent probes for revealing an amplification of the target nucleic acid sequence(s), the composition comprising:
reagents and nucleic acid amplification enzymes, wherein the composition further comprises a polymerisable reagent, which polymerises under the influence of a stimulation selected from among a thermal stimulation and a light stimulation, so as to form a hydrogel locally encapsulating each target nucleic acid sequence when said composition is exposed to said stimulation.

2. The composition according to claim 1, wherein the polymerisable reagent is selected from among a photosensitive reagent that polymerises when exposed to light, and a heat-sensitive reagent that polymerises when exposed to a threshold temperature.

3. The composition according to claim 1, wherein the polymerisable reagent is selected from among the class of PEGs and other water-soluble polymers compatible with PCR.

4. The composition according to claim 1, wherein the polymerisable reagent is selected from among the class of PEGs and other water-soluble polymers compatible with PCR, capable of photosensitive or heat-sensitive reactions such as radical, ionic, dimerisation or Diels-Aider reactions.

5. The composition according to claim 1, wherein the polymerisable reagent is present in a content from 2% to 25%, preferably from 5% to 15%, by weight per unit volume (w/V).

6. The composition according to claim 1, further comprising an initiator agent selected from among a photo-initiator and a thermo-initiator, in a content from 0.02% to 1% by weight per unit volume (w/V).

7. The composition according to claim 1, comprising the specific primers of the target nucleic acid sequence(s), as well as fluorescent probes for revealing an amplification of the target nucleic acid sequence(s).

8. The composition according to claim 1, wherein the composition remains liquid before application of said stimulation for a time period comprised between several hours and several months, and said composition forms the hydrogel after application of said stimulation within a time period shorter than or equal to 10 minutes.

9. A method for amplifying nucleic acid sequences comprising the following steps:
(i) providing a composition according to claim 1;
(ii) mixing said composition with a biological sample to be analysed;
(iii) applying a stimulation selected from among a thermal stimulation and a light stimulation so as to polymerise the mixture of step (ii) to obtaining a hydrogel;
(iv) exposing the hydrogel to an amplification reaction of acid sequences.

10. The method according to claim 9, wherein the provision step (i) is maintained for a time period comprised between several hours and several months.

11. The method according to claim 9, wherein said reaction is a polymerase chain reaction.

12. The amplification method according to claim 9, wherein step (iii) of applying a stimulation includes exposing said composition to a UV light or a threshold temperature.

13. The amplification method according to claim 9, wherein the hydrogel is obtained within a time period shorter than or equal to 10 minutes.

14. The amplification method according to claim 9, wherein step (iii) includes a reaction selected from among the group formed by a bio-orthogonal thiol-ene reaction and a dimerisation reaction.

15. The composition according claim 6, wherein the photo-initiator is lithium phenyl-2,4,6-trimethylbenzoylphosphinate (LAP), and the thermo-initiator is 2,2′-azobis [2-(2-imidazolin-2-yl) propane] dihydrochloride.
